SEQUENCE LISTING (4 sequences)

SEQ ID NO: 1 - DNA, 22 bp, Artificial
  Primer
cacatagaagcctagcccacac

SEQ ID NO: 2 - DNA, 22 bp, Artificial
  Primer
gcttgtcctgtgcagctgggac

SEQ ID NO: 3 - DNA, 20 bp, Artificial
  Primer
tcctcaaagatgctcattag

SEQ ID NO: 4 - DNA, 20 bp, Artificial
  Primer
gtaactcactcatgcaaagt

(19) United States
(12) Patent Application Publication
(10) Pub. No.: US 2007/0059289 A1
(43) Pub. Date: Mar. 15, 2007

(54) Methods of improving cardiac function and attenuating and/or preventing cardiac remodeling with Hsp20

(76) Inventors: Evangelia Kranias, Cincinnati, OH (US); Guo-Chang Fan, Cincinnati, OH (US); Guoxiang Chu, Mason, OH (US)

Correspondence Address: DINSMORE & SHOHL, LLP, 1900 CHEMED CENTER, 255 EAST FIFTH STREET, CINCINNATI, OH, 45202, US

(21) Appl. No.: 11/503,706

(22) Filed: Aug. 14, 2006

Related U.S. Application Data
(60) Provisional application No. 60/707,704, filed on Aug. 12, 2005.

(51) Int. Cl. A61K 48/00; A61K 38/17; A61K 9/127

(52) U.S. Cl. 424/93.2; 514/12; 514/44; 424/450; 977/802

(57) ABSTRACT

Methods of improving cardiac function and/or methods for attenuating and/or prevention cardiac remodeling in an individual's heart comprising administering to an individual an effective amount of Heat-Shock Protein Hsp20 or an agent that increase the levels of and/or the activity of Hsp20 are provided.

### RELATED APPLICATION

This application claims priority under 35 U.S.C. §119 of U.S. Provisional Application Ser. No. 60/707,704 filed Aug. 12, 2005.

### GOVERNMENT INTERESTS

This invention was made, at least in part, with funds from the Federal Government, awarded through grant numbers HL-26057, HL-64018 and HL-52318. The U.S. Government therefore has certain acknowledged rights to the invention.

### FIELD OF THE INVENTION

The present invention is directed towards methods of improving cardiac function and/or methods for attenuating and/or prevention of cardiac remodeling in an individual's heart. The invention is also directed to methods of enhancing stem cell survival.

### BACKGROUND OF THE INVENTION

Heart disease is a major cause of death and disability. For example, ischemic heart disease causes approximately one third of all deaths in men and approximately one quarter of all deaths in women. This detriment reflects lack of effective therapies, targeted to the underlying biological processes within diseased and/or ischemic cardiomyocytes. In the heart, transient ischemia followed by reperfusion (ischemia/reperfusion, I/R) induces necrosis and apoptosis, leading to myocardial dysfunction. Preservation of cardiac function, attenuation and/or prevention of cardiac remodeling and reduction of the infracted area after I/R depends on critical adaptive responses. Accordingly, it would be advantageous to develop therapies for improving cardiac function, attenuating and/or preventing of cardiac remodeling, and/or reducing or preventing I/R injury in the heart.

Recent experimental results have suggested the possibility of regenerating damaged myocardium using stem cells, such as adult bone-marrow-derived mesenchymal stem cells (MSCs). However, a major limitation to the efficacy of stem cell therapy is the poor viability of the transplanted cells. Indeed, the functional improvement from stem cell therapy has been quite modest. A high level of engrafted cell death occurs within four days after grafting into injured hearts. Furthermore, regenerated tissue from stem cells does not survive repeated bouts of ischemia. Therefore, cytoprotection for one week is critical for improving the efficiency of cell therapy, and genetic modification of stem or progenitor cells may represent an important strategic advancement in regenerative medicine.

### SUMMARY OF THE INVENTION

The present invention is directed to methods of improving cardiac function in an individual's heart. The methods comprise administering to the individual an effective amount of Heat-Shock Protein Hsp20or an agent that increases the level of and/or the activity of Hsp20to improve the function in the heart.

The present invention is also direct to methods of attenuating and/or preventing cardiac remodeling in an individual's heart. The methods comprise administering to the individual an effective amount of Heat-Shock Protein Hsp20or an agent that increases the level of and/or the activity of Hsp20 to attenuate and/or prevent cardiac remodeling.

The present invention is further directed to methods of enhancing stem cell survival. The methods comprise modifying a stem cell with Hsp20 to enhance the survival of the stem cells.

### BRIEF DESCRIPTION OF THE FIGURES

The following Detailed Description may be more fully understood in view of the Figures, in which:

FIG. 1 illustrates the generation and characterization of cardiac-specific Hsp20 TG mice. a, Diagram of Hsp20 TG construct. TG mice are generated by use of mouse cardiac Hsp20 cDNA (mcHsp20) under control of α-myosin heavy chain promoter (αMHCp), a 600-bp DNA fragment containing human growth hormone polyadenylation sequences (hGHpA) ligated 3′ to 500-bp mcHsp20 cDNA open reading frame. Fragment containing mcHsp20 /hGHpA is then ligated downstream from 5.5 kb of mouse cardiac-specific αMHC promoter. Positions of PCR primers used for genotyping are indicated by arrows. b, Genotypic analysis of genomic DNA from WT (−) and TG (+) mice. Control PCR is set up to amplify a 350-bp fragment of TSH-β. c, Ventricular sections from 3-month-old mice stained with hematoxylin and eosin (magnification, ×400) indicate no apparent morphological/pathological abnormalities in TG hearts. d, Aliquots (20 μg) of different tissue homogenates from WT and TG mice are subjected to Western blotting with an anti-Hsp20 antibody. e, Quantitative immunoblotting analysis show that there is a 10-fold increase in Hsp20 protein level in TG hearts relative to WT hearts (1.0). f, Increased Hsp20 expression did not alter Hsp25 and αβ-crystallin protein levels.

FIG. 2 illustrates that Hsp20 overexpression improves postischemic cardiac performance in isolated perfused hearts. After 30 minutes of stabilization with KH buffer, hearts are subjected to 45 minutes of global ischemia and 2 hours of reperfusion. a, Left ventricular developed pressure (LVDP) during reperfusion is higher in TG than in WT hearts. b and c, Recovery of postischemic contractile function (±dP/dt) is significantly improved in TG hearts (n=15, WT; n=12, Hsp20 TG; *P<0.05 vs WT).

FIG. 3 illustrates that Hsp20 overexpression attenuates I/R-induced necrosis and apoptosis. Hsp20 TG hearts subjected to 45 minutes of no-flow ischemia followed by 2 hours of reperfusion exhibit significantly decreased (a) total LDH efflux (U/g wet heart wt) in coronary effluent, collected during first hour of reperfusion; (b) DNA fragmentation; and (c) positive number of TUNEL-stained cells (dark brown), indicated by arrows (n=8), compared with WT hearts (n=6). *P<0.05 vs WT.

FIG. 4 illustrates that Hsp20 overexpression alleviates I/R injury. a-d, Representative trichrome-stained myocardial sections are from ex vivo postischemic hearts. Contraction bands (long arrows) and vacuoles (short arrows), all signs of cellular damage, is present in WT (a and c) but not TG (b and d) hearts. e, Cardiac specific overexpression of Hsp20 significantly reduce ex vivo myocardial infarct size, expressed as a percentage of area at risk, after 45 minutes of no-flow ischemia and 120 minutes of reperfusion (5 to 6 slices per heart; n=6, TG; n=8, WT; *P<0.0l vs postischemic WT hearts). f, Hsp20 overexpression greatly reduces in vivo myocardial infarct size after 30 minutes of myocardial ischemia, via LAD occlusion (top left), followed by 24 hours of reperfusion. Hearts are infused through aorta with 1% TTC, followed by 5% phthalo blue, and then cut transversely into slices 2 to 3 mm thick. Area of necrosis and area at risk are quantified as previously described 28 (n=7, TG; n=11, WT; *P<0.001 vs postischemic WT hearts). Region at risk is not significantly different between groups (TG, 52.9±1.5; WT, 45.6±3.2; P<0.05). Infarct sizes before normalization are TG, 4.3±0.5; WT, 8.8±1.2; P<0.05.

FIG. 5 illustrates the effects of Hsp20 overexpression on apoptosis-related proteins. In ex vivo WT hearts, protein levels of Bcl-2 (a) are significantly decreased, whereas Bax (b) levels are significantly increased, after 45 minutes of no-flow ischemia, followed by 120 minutes of reperfusion. These proteins show no alterations in TG hearts. c, Ratio of Bcl-2/Bax protein levels in post-ischemic/reperfused TG hearts are significantly higher than in WT hearts. d and e, Hsp20 coimmunoprecipitated with Bax but not with Bcl-2. Mouse heart homogenates are immunoprecipitated with anti-Hsp20 (d) or anti-Bax antibodies (e). Proteins are separated on SDS-PAGE gels and probed with anti- Bax (d1) or anti-Hsp20 antibodies (e1). Then, membrane (d1) is stripped and re-probed with anti-Bcl-2(d2); subsequently, this membrane (d2) is re-stripped and re-probed with anti-Hsp20 (d3). Similarly, membrane (e1) is stripped and re-probed with anti-Bax antibodies (e2). Preimmunoprecipitated heart homogenate is used as positive control (+), and immunoprecipitate without Hsp20 or Bax antibodies is used as negative control (−). f, Caspase-3 activity is significantly lower in TG hearts than in WT hearts after I/R. *P<0.05 vs post-ischemic/reperfused WT hearts; n=6 in each group. g-j, Hsp20 phosphorylation is analyzed by use of 2D gel electrophoresis.

Homogenates are obtained from pre-ischemic/reperfused (g and i) and post-ischemic/ reperfused (h and j) WT and TG hearts that are subjected to occlusion of LAD artery for 30 minutes and reperfusion for 24 hours. Homogenates are extracted by use of a solubilization solution of 7 mol/L urea, 2 mol/L thiourea, 4% CHAPS, 20 mmol/L DTT, 20 mmol/L spermine, and 1 mmol/L PMSF. Extract is centrifuged (100 000 g, 1 hour, at 10° C.), and 500 μg from each preparation is separated by 2D electrophoresis, using isoelectric focusing strips (Immobiline Drystrips, 18 cm, pH 4 to 7). 2D gels are Sypro Ruby-stained, and protein spots corresponding to phosphorylated (pI of 5.5) or nonphosphorylated (pI of 5.7) Hsp20 is further confirmed by Western blotting and LCMS/ MS analysis (data not shown).

### DETAILED DESCRIPTION OF THE INVENTION

Heat-shock protein (Hsp) synthesis arises transiently as a tool to protect cellular homeostasis after exposure to heat and a wide spectrum of stressful and potentially deleterious stimuli. Hsps have been implicated as mediators of myocardial protection, particularly in experimental models of ischemia and reperfusion injury. Hsps have also been shown to render cardioprotection from stress-induced injury. The cardioprotective effects of Hsp 70 have been shown in isolated adult feline cardiomyocytes, rabbit hearts after adenovirus-mediated gene transfer, and transgenic (TG) mouse hearts after global or regional ischemia. Recently, protection during myocardial ischemia has also been shown for the small heat-shock proteins Hsp27 and αβ-crystallin; however, little is known about the role of another small Hsp, Hsp20 , which shares considerable sequence homology with Hsp27 and αβ-crystallin, in cardioprotection against ischemic injury.

The small Hsp, Hsp20 , was initially identified as a member of the crystallin Hsp family from skeletal muscle. It provides resistance to heat treatment in Chinese hamster ovary cells, and its expression levels are elevated on heat pretreatment of swine carotid artery, insulin exposure of skeletal muscle, and β-agonist stimulation of cardiomyocytes. Interestingly, Hsp20 has been shown to regulate vasodilation and suppress platelet aggregation. Moreover, the recent studies of the inventors have indicated that adenoviral gene transfer of Hsp20 in isolated cardiomyocytes improved contractile function and protected against β-agonist-mediated apoptosis.

Increasing evidence has shown that several Hsps have antiapoptotic roles, and overexpression of Hsp27, αβ-crystallin, Hsp32 (HO-1), and Hsp70 in the heart can attenuate I/R injury and improve cardiac function; however, the Hsp20 studied here is different from the other Hsps in its unique protein kinase A/protein kinase G phosphorylation site RRAS (phosphorylation of the Ser-16 site significantly increases the contractility in cardiomyocytes; unpublished data), regulatory activities of vasorelaxation, and platelet aggregation. Furthermore, previous studies have shown that Hsp20 is translocated to actin filaments on stress, suggesting its cytoskeletal stabilizing function in cardiomyocytes. Taken together, these properties of Hsp20 suggest that it may benefit the ischemic heart at multiple levels.

To further define the functional significance of Hsp20 in vivo and its potential protective mechanisms, the inventors generated a TG mouse model with cardiac-specific overexpression of Hsp20 . The findings demonstrate that increased Hsp20 expression in the heart improves cardiac function. Accordingly, the inventors have determined methods for improving cardiac function in an individual's heart. The methods comprise administering to the individual an effective amount of Hsp20 or an agent that increases the level of and/or the activity of Hsp20 to improve cardiac function in the heart. The inventors have further determined that these methods may also be employed to improve the survival rate of not only the individual, but also the survival rate of a tissue and/or a cell. As used herein, “individual”, is intended to refer to an animal, including but not limited to humans, mammals, and rodents.

One skilled in the art will appreciate the various means in which the cardiac function of an individual's heart may be improved using the methods of the present invention. In one embodiment, cardiac function is improved by attenuating and/or reducing hypertrophy in the individual's heart. In another embodiment, cardiac function is improved by enhancing cardiac contractility in the individual's heart. In yet another embodiment, cardiac function is improved by attenuating and/or preventing the development or the time course of heart failure in an individual.

One skilled in the art will appreciate that the Hsp20 administered to the individual may comprise full-length Hsp20 , a Hsp20 fragment or a combination thereof. As used herein, “Hsp20 fragment” refers to any fragment of the full-length Hsp20 that has the same activity as full-length Hsp20 , or is a chemical or structural analogue of the full-length Hsp20 . Agents that increase the levels of and/or the activity of Hsp20 may also be employed in the present invention to improve cardiac function. One skilled in the art will appreciate the various agents that increase the level and/or the activity of Hsp20 , any of which may be employed herein. One skilled in the art will also appreciate the various effective amounts of Hsp20 or an agent that increases the level of and/or the activity of Hsp20 which may be administered to an individual for improving cardiac function and/or attenuating and/or preventing cardiac remodeling in the heart, any of which may be employed herein.

Hsp20 and/or an agent that increases the level of and/or the activity of Hsp20 may be administered by any methods known to one of ordinary skill in the art. In one embodiment, the administration of Hsp20 and/or an agent that increases the level of and/or the activity of Hsp20 comprises intravenous, intradermal, subcutaneous, oral, transdermal, transmucosal, or a combination thereof. In another embodiment, for nucleic acid delivery of Hsp20 and/or an agent that increases the level of and/or the activity of Hsp20 the administration comprises a viral vector, liposome, a non-viral delivery system or a combination thereof.

As illustrated in the Example, cardiac function may also be improved by reducing and/or preventing ischemia and reperfusion (I/R) injury in the individual's heart by administering Hsp20 or an agent that increases the level of and/or the activity of Hsp20 . In one embodiment, cardiac function of the individual is improved after the I/R injury as compared with an individual not administered Hsp20 or an agent that increases the level of and/or the activity of Hsp20 . In another embodiment, the infarction size is reduced after the I/R injury as compared to an individual not administered Hsp20 or an agent that increases the levels of and/or the activity of Hsp20 . In yet another embodiment, there is less damage to structural components of the heart after I/R injury as compared to an individual not administered Hsp20 or an agent that increases the level of and/or the activity of Hsp20 .

Recently, Hsp60 and αβ-crystallin have also been shown to be complexed with the proapoptotic protein Bax. Under stress conditions, these Hsps and Bax dissociate, whereupon Bax translocates to the mitochondria to participate in apoptosis, suggesting a role for Hsps upstream of caspase activation; however, Hsp70 is found to protect cells from death induced by enforced expression of caspase-3, suggesting that protection by Hsp70 may occur downstream of caspase activation. In addition, Hsp70 has been reported to interfere with apoptosis by a direct interaction with Apaf-1. Moreover, Hsp27 has been reported to directly bind to cytosolic cytochrome c and sequester it from Apaf-1. Thus, different Hsps may act via different mechanisms to prevent cell death. The data presented in the Example demonstrates that Hsp20 prevents I/R-induced apoptosis, possibly through the Bax-caspase pathway. While not wishing to be bound by theory, it is believed that apoptosis-related proteins, such as Bcl-2 and Bax, are stabilized after the I/R injury and carpase-3 activity is decreased after the I/R injury.

In addition to improving cardiac function, the inventors have determined methods of attenuating and/or preventing cardiac remodeling in an individual heart. The methods comprise administering to the individual an effective amount of Hsp20 or an agent that increases the levels of and/or the activity of Hsp20 to attenuate and/or prevent cardiac remodeling in the individual. Examples of cardiac remodeling include, but are not limited to, hypertrophy, heart failure progression or a combination thereof. In one embodiment, administration of Hsp 20 or an agent that increases the level of and/or the activity of Hsp20 improves the maintenance of muscle integrity, for example, during I/R injury, as evidenced in the Example by the presence of less damaged myofibril in the TG hearts after an I/R injury.

While not wishing to be bound by theory, because there is strong evidence that cytoskeletal injury plays a crucial role in the pathogenesis of myocardial ischemic injury, it is plausible that Hsp20 translocates from the soluble fraction of cardiomyocytes to the insoluble fraction after I/R, leading to protection of the collapsed intermediate filament network or cytoskeletal protein damage.

These cardioprotective effects may also be associated with increased Hsp20 phosphorylation, consistent with previous reports on enhanced interaction of phosphorylated Hsp20 with actin and actinin, which further stabilizes the microfilaments. In addition, while not wishing to be bound by theory, the inventors believe that other mechanisms may include ASK1, P13K-Akt and protein phosphatase 1. Accordingly, the inventors' findings demonstrate that increased expression of Hsp20 and its accompanied phosphorylation protects the heart, leading to restoration of cardiac function and reduced infarction.

Cardiac function may be improved and/or cardiac remodeling may be attenuated and/or prevented in an individual that has or is at risk of developing a heart disorder. As used herein, “heart disorder” refers to a structural or functional abnormality of the heart that impairs its normal functioning. One skilled in the art will appreciate the various types of heart disorders. For example, heart disorder includes, but is not limited to, heart failure, ischemia, myocardial infarction, congestive heart failure or any combination of heart disorders. In one embodiment, the heart of the individual that has or is at risk of developing a heart disorder has full functional recovery after administration of Hsp20 or an agent that increases the level of and/or the activity of Hsp20. In another embodiment, the integrity of the muscle of the heart of the individual that has or is at risk of developing a heart disorder is maintained after administration of Hsp20 or an agent that increases the level of and/or the activity of Hsp20.

While not wishing to be bound by theory, the inventors also believe that stem cells, including but not limited to adult stem cells, embryonic stem cells, bone-marrow-derived stem cells, such as mesenchymal stem cells (MSCs), or combinations thereof, modified with Hsp20 will become more resistant to apoptosis in vitro and in vivo. When injected into infarcted hearts, stem cells modified with Hsp20 will limit ventricular remodeling and improve cardiac function, leading to more efficient healing compared with control stem cells, suggesting an ideal combination of cell and gene therapy for myocardial repair and regeneration. Clinical benefits of administering Hsp20 may also be employed with using adenoviral or adenovirusassociated viral gene transfer in vivo or engraft Hsp20 -modified stem cells after myocardial infarction may further elucidate the potential clinical benefits of Hsp20

### EXAMPLES

Methods

To investigate whether overexpression of Hsp20 exerts protective effects in both ex vivo and in vivo ischemia/reperfusion (I/R) injury, the inventors generate a transgenic (TG) mouse model with cardiac-specific overexpression of Hsp20 (10-fold).

Generation of TG Mouse Model

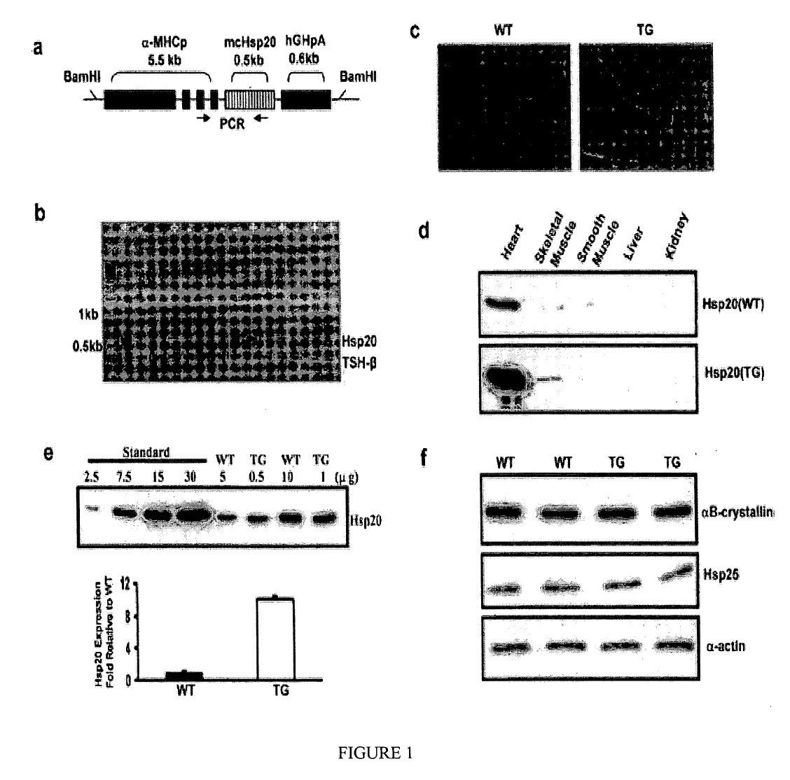

TG mice are generated by using mouse cardiac Hsp20 cDNA under the control of the α-myosin heavy chain promoter (αMHCp) (FIG. 1a). The TG lines used in this study have a 10-fold overexpression of Hsp20. Routine genotyping is performed by polymerase chain reaction (PCR) with the use of an upper primer from the αMHC promoter (5′-CACATAGAAGCCTAGCCCACAC-3′) (SEQ ID NO: 1) and a lower primer from the mcHsp20 cDNA (5′-GCTTGTCCTGTGCAGCTGGGAC-3′) (SEQ ID NO:2) to amplify a 600-bp fragment spanning the junction between the αMHC promoter and mcHsp20 cDNA. The control PCR is set up to amplify a 350-bp fragment of TSH-β using an upstream primer: 5′-TCCTCAAAGATGCTCATTAG-3′(SEQ ID NO: 3) and a downstream primer: 5′-GTAACTCACTCATGCAAAGT-3′(SEQ ID NO: 4) (FIG. 1b).

Immunoblotting

Heart homogenates are analyzed by standard Western blotting to compare Hsp20 , Hsp25, αβ-crystallin, Bcl-2, Bax, actin, and α-actin levels. Binding of the primary antibody is detected by peroxidaseconjugated secondary antibodies and enhanced chemiluminescence (Amersham), and bands are quantified with densitometry. Antibodies Hsp20 (Research Diagnostics Inc), Hsp25 (Affinity BioReagents Inc), αβ-crystallin (Calbiochem), Bcl-2(C-2), Bax (B-9), actin, and α-actin (Santa Cruz Biotechnology Inc).

Immunoprecipitation

Association of Hsp20 with Bax and Bcl-2 is studied using Dynabeads Protein G (Dynal Biotech) according to the manufacturer's instructions. Briefly, cardiac homogenate from wild-type (WT) heart is precleared by incubating with the beads for 1 hour at 4° C. to minimize nonspecific binding. Fresh beads are washed with 0.1 mol/L sodium phosphate buffer and then coated with anti-Hsp20 or anti-Bax antibody. The bound antibody is crosslinked to the beads using 20 mmol/L dimethyl pimelidate in a 0.2 mol/L triethanolamine solution. The precleared homogenate is then added to the crosslinked beads, and binding is mediated at 4° C. for 1 hour. Finally, the proteins are eluted off (using 0.1 mol/L citrate), and their identity is determined by immunoblotting.

Global Ischemia Ex Vivo

The cellular and functional responses to I/R are assessed in mice by using an isolated perfused heart model as previously described. Male adult mice (12 to 14 weeks old) are anesthetized intraperitoneally (IP) with pentobarbital sodium (50 mg/kg). Hearts are rapidly excised and mounted on a Langendorff apparatus, perfused with Krebs-Henseleit buffer (noncirculating), and stabilized for 30 minutes, and then the hearts are subjected to 45 minutes of no-flow global ischemia and 2 hours of reperfusion. A fluid-filled balloon made of plastic film is inserted into the left ventricle via the mitral valve and inflated to yield a left ventricular end-diastolic pressure of 10 mm Hg. The balloon is attached via polyethylene tubing (PE50) to a pressure transducer connected to a Heart Performing Analyzer (Micro-Med), and continuous left ventricular pressure is measured. A bipolar electrode (NuMed) is inserted into the right atrium, and atrial pacing is performed at 400 bpm with a Grass S-5 stimulator.

Pacing is stopped during ischemia and restarted at reperfusion. After reperfusion, the hearts are weighed, frozen, and cut into 2-mm-thick slices parallel to the atrioventricular groove. The slices are thawed and stained by incubation in 1% triphenyl tetrazolium chloride (TTC) solution in phosphate buffer (Na₂HPO₄ 88 mmol/L, NaH₂PO₄ 1.8 mmol/L) at 37° C. for 10 to 20 minutes as previously described. The area of infarction, risk zone, and nonrisk myocardium are determined by planimetry of each slice.

Lactate Dehydrogenase Release and TUNEL Assays

In addition to cardiac function, cardiac injury is assessed by measuring lactate dehydrogenase (LDH) release. Perfusion effluent is collected every 15 minutes of preischemia and also during reperfusion. Total LDH released from the heart is determined using a CytoTox 96 assay (Promega) and expressed as units per gram of wet heart weight.

For terminal dUTP nick end-labeling (TUNEL) assays, hearts are removed from the apparatus after I/R, and the atrial tissue is dissected away. The ventricles are fixed in 10% buffered formalin and later embedded in paraffin according to standard procedures, and sections 3 μm thick are obtained to perform TUNEL assays using the ApopTag Plus Peroxidase In situ Apoptosis Detection Kit (Chemicon) according to the manufacturer's instructions. TUNEL-positive myocytes are determined by randomly counting 10 fields of the midventricular section and are expressed as a percentage of the total cardiomyocyte population.²⁵

DNA Fragmentation

Heart samples are Dounce-homogenized in 2.5 vol of cell lysis buffer (RIPA, in μmol/L: 1 DTT and 50 PMSF) and centrifuged at 13 000 g for 10 minutes, and supernatant (100 μg) from each heart is used in a photometric enzyme immunoassay for the quantitative determination of cytoplasmic histone-associated DNA fragments (mononucleosomes and oligonucleosomes) of programmed cell death using the commercial assay kit Cell Death Detection ELISAPLUS (Roche). Results are normalized to the standard provided in the kit and expressed as folds of increase over control.

Caspase-3 Activity

To detect caspase-3 activity, 200 μg of lysate from each heart is combined with fluorogenic caspase-3 substrate, diluted to 300 mg/L in caspase assay buffer (250 mmol/L PIPES, 50 mmol/L EDTA, 2.5% CHAPS, and 125 mmol/L DTT), and measured immediately in a fluorometer. Measurements are repeated every 10 minutes for 1 hour, the slope of fluorescent units per hour is calculated, and values are compared with known standards to determine enzymatic activity.

Regional Ischemia In Vivo

Mice weighing 25 to 30 g are anesthetized with sodium pentobarbital (90 mg/kg IP), intubated with PE 90 tubing, and ventilated by use of a mouse miniventilator (Harvard Apparatus) with room air supplemented with oxygen. The respiratory rate is 100 to 105 breaths per minute, and PO₂, PCO₂, blood pH, and body temperature are maintained within normal limits throughout the procedure as previously described.²⁸ ECG electrodes are placed subcutaneously, and data are recorded with a Digi-Med Sinus Rhythm Analyzer (Micro-Med). A lateral thoracotomy (1.5-cm incision between the second and third ribs) is performed to provide exposure of the left anterior descending coronary artery (LAD), while avoiding rib and sternal resection, retraction, and rotation of the heart. Vascular bundles in the vicinity are coagulated by use of a microcoagulator (Medical Industries). An 8-0 nylon suture is placed around the LAD at 2 to 3 mm from the tip of the left auricle, and a piece of soft silicon tubing (0.64 mm ID, 1.19 mm OD) is placed over the artery. All mice are subjected to a 30-minute coronary occlusion by tightening and tying the suture. Ischemia is confirmed by visual observation (cyanosis) and continuous ECG monitoring. After 24 hours of reperfusion, the aorta is cannulated, and the heart is perfused with 1% TTC (37° C., 60 mm Hg) as previously described. The occluder, which had been left in place, is retied, and the heart is perfused with 5% phthalo blue. Hearts are transversely cut into 5 to 6 sections, with 1 section made at the site of the ligature. Infarct sizes are determined and expressed as a percentage of the region at risk.

Statistical Analysis

Data are expressed as mean ±SEM. Statistical analysis is performed using a 2-tailed Student t test for unpaired observations and ANOVA for multiple comparisons. Values of P<0.05 are considered statistically significant.

Results

Generation of Hsp20 TG Mice

The TG mice that carry the mouse cardiac Hsp20 cDNA under the control of the αMHC mouse promoter is generated (FIG. 1, a and b). All Hsp20 TG mice are healthy and show no apparent cardiac morphological or pathological abnormalities (FIG. 1c). Western blot analysis (FIG. 1d) of tissue homogenates reveal that Hsp20 is expressed predominantly in the cardiac and in much lower abundance in skeletal and smooth muscles of WT mice. Transgenesis result in a 10-fold increase of the Hsp20 protein level in the Hsp20 protein level in the TG hearts (FIG. 1e). Cardiac overexpression of Hsp20 does not alter the expression of other small Hsps, such as Hsp25 and αBar-crystallin, in the heart (FIG. 1f).

Improvement in the Postischemic Recovery of Function
Baseline Functional Parameters In Langendorff-Perfused Isovolumically
Contracting Mouse Hearts

| | HR, bpm | BW, g | HW, mg | LVDP, mm Hg | +dP/dt, mm Hg/s | −dP/dt, mm Hg/s | Outflow, mL/min |
|---|---|---|---|---|---|---|---|
| TG, n = 8 | 354 ± 15 | 27.4 ± 1.2 | 174 ± 14 | 83 ± 6* | 4429 ± 149* | 2370 ± 128* | 1.3 ± 0.2 |
| WT, n = 9 | 363 ± 20 | 27.8 ± 0.9 | 171 ± 12 | 69 ± 4 | 3311 ± 185 | 1891 ± 100 | 1.4 ± 0.3 |

HR indicates heart rate; BW, body weight; HW, heart weight; LVDP, left ventricular developed pressure; +dP/dt, rate of contraction; and −dP/dt, rate of relaxation.
*P < 0.05 vs corresponding WT group.

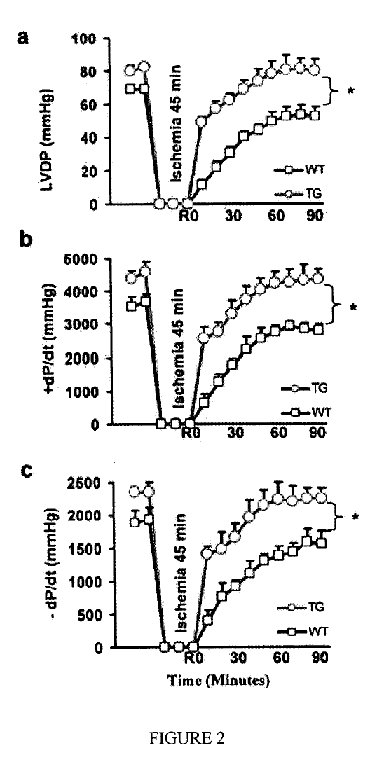

Because acute expression of Hsp20 in rat cardiomyocytes is protective against apoptosis, it is examined whether increased in vivo expression of Hsp20 protects against postischemic injury. To exclude the involvement of inflammatory components on reperfusion, an isolated perfused heart preparation is used Body weights and heart weights of the mice used in these studies are similar between the TG and WT groups (Table). Hearts are stabilized for 30 minutes, and baseline function is measured. Hsp20 overexpression results in an increased contractile function under basal conditions (Table), consistent with the inventors' previous report using adenovirus-mediated Hsp20 gene transfer in cardiomyocytes. 20 Hearts are then subjected to 45 minutes of global ischemia and 2 hours of reperfusion. During reperfusion, the TG hearts exhibit significantly better functional recovery than the WT hearts (FIG. 2). It is noteworthy that recovery of left ventricular develop pressure and the rates of contraction(+dP/dt) and relaxation (−dP/dt) are 54±5% and 59±4%, respectively, in the TG hearts and only 20±3% and 21±3%, respectively, in WT hearts after 10 minutes of reperfusion. Most importantly, these parameters are completely recovered after 1 hour of reperfusion in TG hearts, whereas WT hearts recovered to 69±6% and 71±4%, respectively, of preischemic values.

Attenuation of I/R-Induced Necrosis and Apoptosis

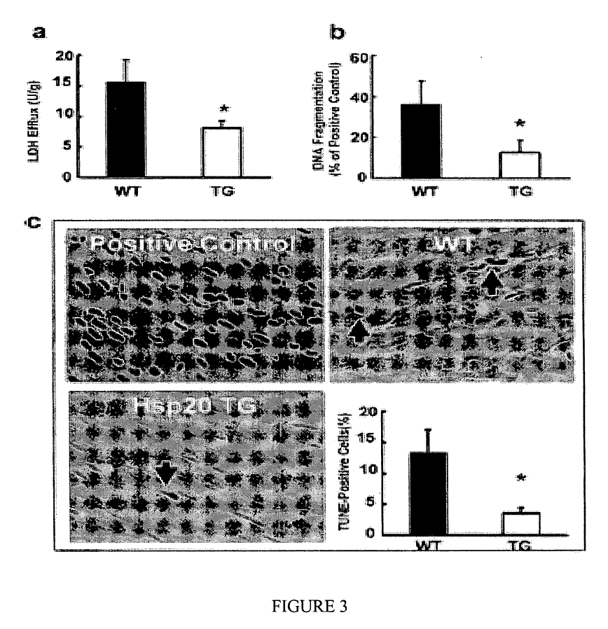

To determine the degree of necrosis in these I/R hearts, the level of LDH released during the first hour of reperfusion after global ischemia is assessed. The total LDH is twice as high in WT hearts compared with TG hearts (FIG. 3a), which indicates reduced necrosis in Hsp20 -overexpressing hearts. Therefore, it is examined whether the functional protection of the Hsp20 TG hearts is related to the antiapoptotic property of small Hsps. Heart lysates from a subset of experimental WT and TG animals are assayed for DNA fragmentation by use of a quantitative nucleosome assay. WT hearts exhibit a 3-fold increase over Hsp20 TG hearts (FIG. 3b). To substantiate the protective effect of Hsp20 via its antiapoptotic action, the TUNEL assays of the hearts are performed. TUNEL-positive cardiomyocytes from the left ventricles of WT animals are 4-fold higher than those in TG animals (FIG. 3c). Thus, both methods of detecting apoptosis (DNA fragmentation and TUNEL assay) demonstrate significant attenuation in TG hearts.

Decreased Myocardial Infarct Size Ex Vivo and In Vivo

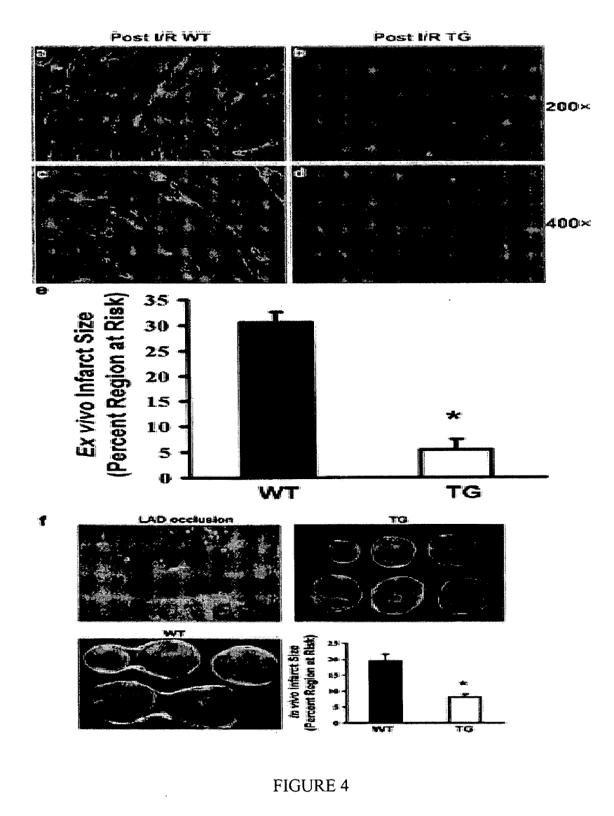

After 45 minutes of no-flow, global ischemia, followed by 120 minutes of reperfusion ex vivo, myocardial infarct size by histochemical and TTC staining is determined. Histological examination of post-ischemic/reperfused WT hearts reveal contraction bands (hypercontracted myofibers) and vacuolizations (FIG. 4, a and c), which are both indicative of cardiomyocyte damage; however, the myofibers are well preserved in Hsp20 TG hearts (FIG. 4, b and d), suggesting that priming the heart with Hsp20 maintains the integrity of the muscle during I/R injury. Myocardial infarct size as a percentage of the area at risk is reduced by 6-fold in TG compared with WT hearts (FIG. 4e). These studies are extended to an in vivo model of 30-minute myocardial ischemia, via coronary artery occlusion, followed by 24-hour reperfusion. Importantly, the infarct region-to-risk region ratio is 8.1±1.1% in TG hearts (n=7), compared with 19.5±2.1% in WT hearts (n=11, P<0.001), under in vivo conditions (FIG. 4f).

Mechanism(s) of Hsp20 Cardioprotective Effects in I/R

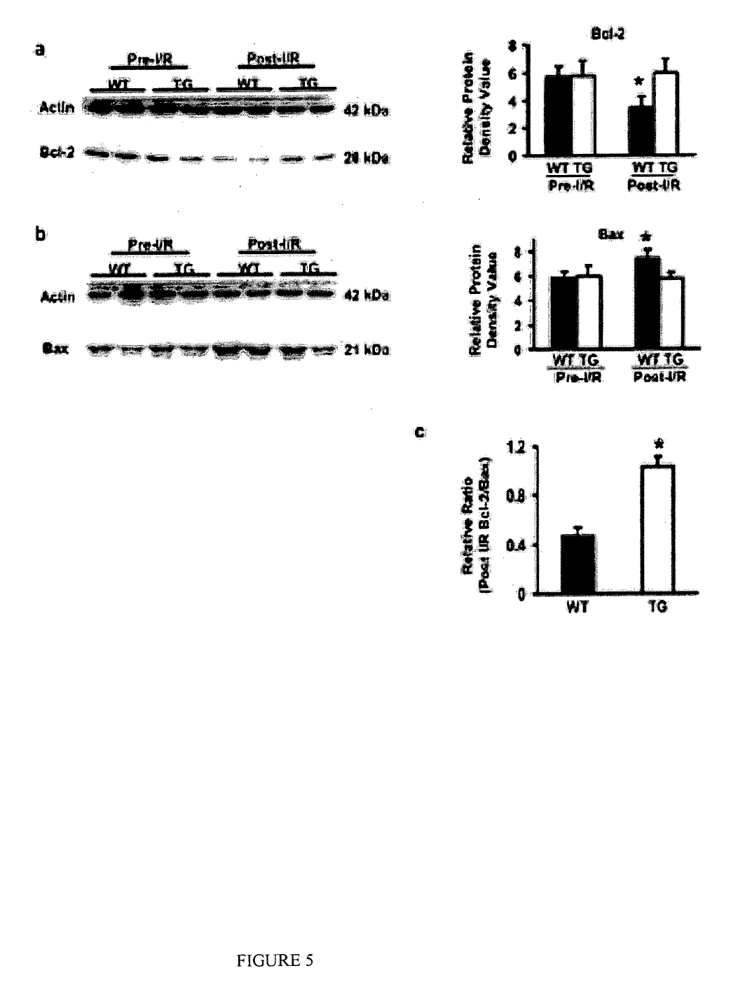

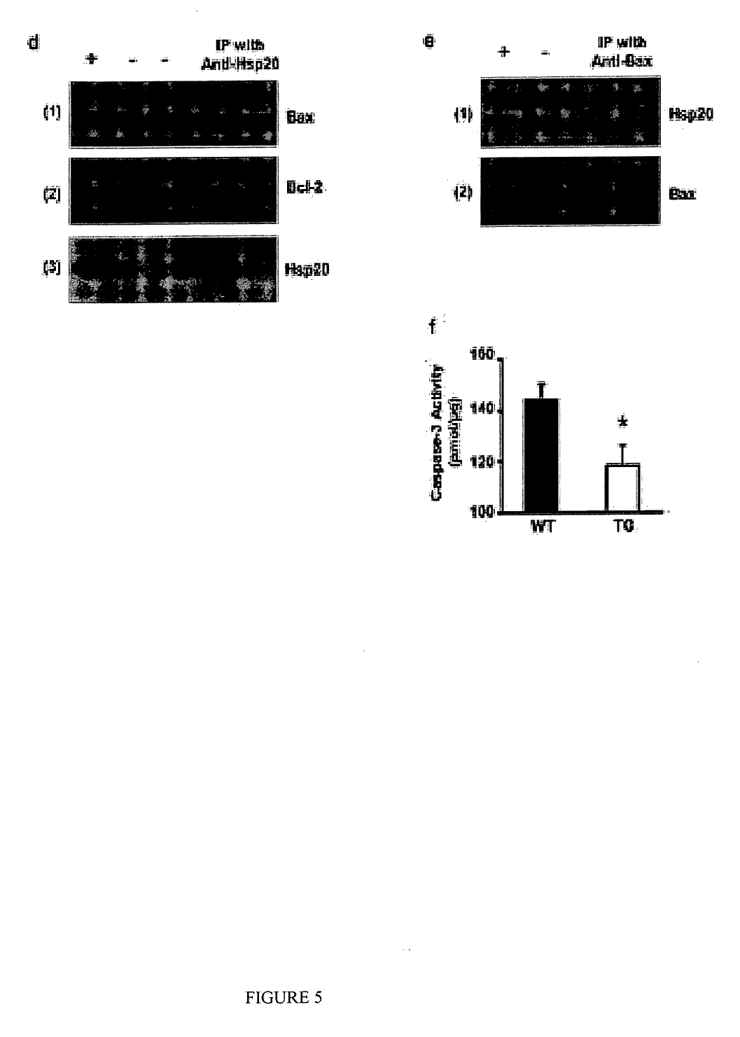

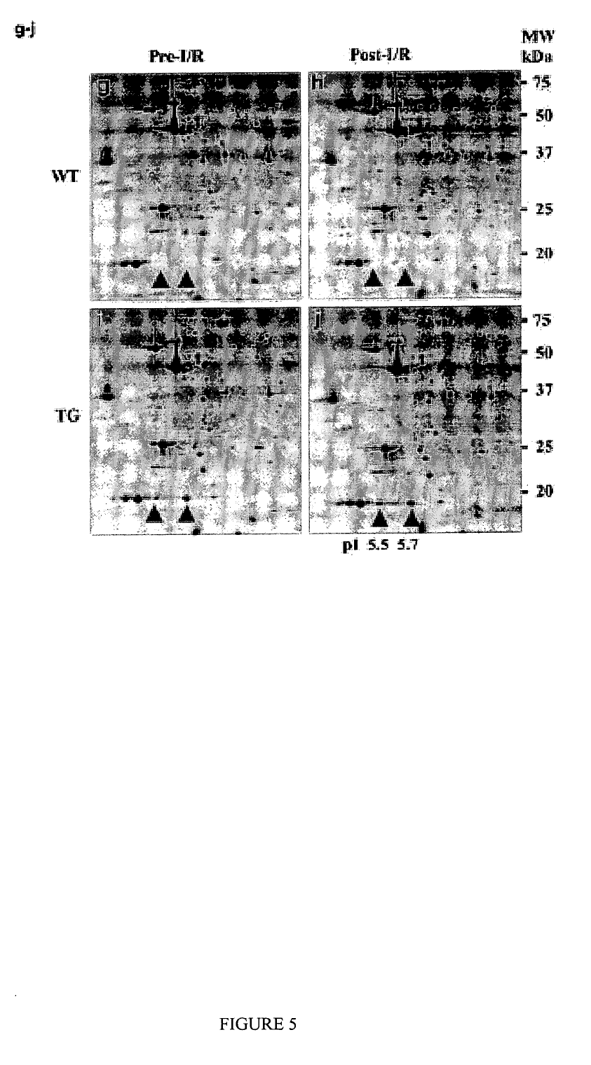

To elucidate the potential mechanism(s) of Hsp20 cardioprotection, Bcl-2 and Bax protein expression levels in hearts before or after I/R is assessed first, because alterations of Bcl-2 and Bax protein levels have been shown in isolated cardiomyocytes after hypoxia/reoxygenation and in hearts during I/R. Overexpression of Hsp20 does not alter the expression levels of either Bcl-2 or Bax (FIG. 5, a and b); however, on I/R, the levels of Bcl-2 are significantly reduced, whereas the levels of Bax are greatly increased, in WT hearts, compared with their preischemic samples (FIG. 5, a and b). Consequently, the relative ratio of Bcl-2 to Bax expression in the TG hearts is more than 2-fold higher, compared with WT hearts after I/R (FIG. 5c). Moreover, immunoprecipitation of homogenates from WT hearts using the Hsp20 antibody reveal that Hsp20 interacted with Bax but not Bcl-2 (FIG. 5d). A reciprocal immunoprecipitation approach using the Bax antibody also demonstrates an association of Bax with Hsp20 (FIG. 5e). Furthermore, the activity of caspase-3 is significantly reduced in TG compared with WT hearts on I/R (FIG. 5f).

Hsp20 has been shown previously to be phosphorylated under β-adrenergic stimulation of isolated cardiomyocytes. To determine whether the protective effects of Hsp20 against I/R injury in vivo may be associated with increases in its phosphorylation levels, the 2D gel electrophoresis is applied. The findings indicate that 16±1% of total Hsp20 is phosphorylated in TG hearts (FIG. 5i). This phosphorylation is significantly increased to 37±2% after 24 hours of reperfusion (FIG. 5j), suggesting that Hsp20 phosphorylation plays an important role in cardioprotection against I/R injury. Similar assessment of altered Hsp20 phosphorylation in WT hearts is not feasible because of the low levels of endogenous Hsp20 expression (FIG. 5, g and h).

In the present study, the inventors discovered the role of Hsp20 on cardioprotection during myocardial ischemia. Interestingly, cardiac-selective overexpression of Hsp20 is associated with full functional recovery and decreased infarct size both ex vivo and in vivo on I/R injury. There are at least 2 mechanisms that underlie cardioprotection in Hsp20 TG mice from I/R injury. The first involves stabilization of the apoptosis-related proteins Bcl-2 and Bax by Hsp20 overexpression (FIG. 5, a-c), whereas WT hearts exhibited decreases in Bcl-2 and increases in Bax levels after I/R, similar to recent findings in vivo. It has been shown previously that overexpression of Bcl-224 or ablation of Bax25 is associated with improved cardiac function, which correlated with a reduction of cardiomyocyte apoptosis, after I/R. In addition, delayed ischemic preconditioning, on phenylephrine treatment of rabbit hearts, resulted in an increased Bcl-2/Bax ratio and a reduction of apoptosis. Thus, preserved Bcl-2 and Bax protein levels may be an important mechanism of inhibiting I/R-induced apoptosis by overexpression of Hsp20.

Importantly, Hsp20 complexed with the protein Bax (FIG. 5, d and e), which possibly prevented the translocation of Bax from the cytosol into the mitochondria during I/R, is demonstrated. As a result, Hsp20 may preserve the integrity of mitochondria, restrict release of cytochrome c, and repress activation of caspase-3. In fact, the data demonstrated decreased caspase-3 activity in post-ischemic/reperfused TG hearts (FIG. 5f), suggesting that cardioprotection from I/R-induced apoptosis may involve inhibition of the conversion of procaspase-3 (p24) to active caspase-3 because of inhibition of Bax translocation (data not shown). It has been shown that caspase-3 expression is increased in association with heart failure and apoptosis in experimental animals. Direct blockade of caspase activation with a peptide inhibitor protects the myocardium against lethal reperfusion injury. In contrast, heart targeted overexpression of caspase-3 in mice has been shown to increase infarct size and depress cardiac function. While not wishing to be bound by theory, it is believed that the decreased activation of caspase-3 might also be one of the cardioprotective mechanisms against I/R-induced injury.

Thus, the inventors have discovered that Hsp20 may provide a new potential therapeutic target for heart disease. Specifically, the findings demonstrate that increased Hsp20 expression in the heart improves cardiac function and/or attenuates and/or prevents cardiac remodeling.

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

What is claimed is:

1. A method of improving cardiac function in an individual's heart comprising administering to the individual an effective amount of Heat-Shock Protein Hsp20 or an agent that increases the level of and/or the activity of Hsp20 to improve cardiac function in the heart.

2. The method of claim 1, wherein cardiac function is improved by attenuating and/or reducing hypertrophy in the individual's heart.

3. The method of claim 1, wherein cardiac function is improved by enhancing cardiac contractility in the individual's heart.

4. The method of claim 1, wherein cardiac function is improved by attenuating and/or preventing the development or the time course of heart failure in an individual.

5. The method of claim 1, wherein cardiac function is improved by reducing and/or preventing ischemia and reperfusion (I/R) injury in the individual's heart.

6. The method of claim 5, wherein cardiac function is improved after the I/R injury as compared with an individual not administered Hsp20 or an agent that increases the level of and/or the activity of Hsp20.

7. The method of claim 5, wherein the infarction size is reduced after the I/R injury as compared to an individual not administered Hsp20 or an agent that increases the level of and/or the activity of Hsp20.

8. The method of claim 5, wherein there is less damage to structural components of the heart as compared to an individual not administered Hsp20 or an agent that increases the level of and/or the activity of Hsp20.

9. The method of claim 5, wherein apoptosis-related proteins are stabilized after the I/R injury.

10. The method of claim 5, wherein carpase-3 activity is decreased after the I/R injury.

11. The method of claim 1, wherein the survival rate of the individual is improved.

12. The method of claim 1, wherein the individual has or is at risk of developing a heart disorder.

13. The method of claim 12, wherein the heart disorder comprises heart failure, ischemia, myocardial infarction, congestive heart failure or a combination thereof.

14. The method of claim 12, wherein the heart of the individual has full functional recovery after administration of Hsp20 or an agent that increases the level of and/or the activity of Hsp20.

15. The method of claim 12, wherein the integrity of the muscle of the heart is maintained after administration of Hsp20 or an agent that increases the level of and/or the activity of Hsp20.

16. The method of claim 1, wherein Hsp20 is phosphorylated.

17. The method of claim 1, wherein the administration comprises intravenous, intradermal, subcutaneous, oral, transdermal, transmucosal, or a combination thereof.

18. The method of claim 1, wherein Hsp20 comprises full-length Hsp20 , a Hsp20 fragment or a combination thereof.

19. The method of claim 18, wherein the administration comprises a viral vector, a liposome, a non-viral delivery system or a combination thereof.

20. A method of attenuating and/or preventing cardiac remodeling in an individual heart comprising administering to the individual an effective amount of Hsp20 or an agent that increases the level of and/or the activity of Hsp20 to attenuate and/or prevent cardiac remodeling in the individual.

21. The method of claim 20, wherein cardiac remodeling comprises hypertrophy, heart failure progression or a combination thereof.

22. A method of enhancing stem cell survival comprising modifying a stem cell with Hsp20 , wherein the survival of the stems cell is enhanced.

23. The method of claim 22, wherein the stem cell comprises an adult stem cell, an embryonic stems cell, a bone marrow derived stem cell or a combination thereof.

24. The method of claim 22, wherein the stem cell modified with Hsp20 attenuates and/or prevents cardiac remodeling and/or to improve cardiac function of the individual's heart.
